(19) United States
(12) Patent Application Publication
(10) Pub. No.: US 2026/0110678 A1
(43) Pub. Date: Apr. 23, 2026

(54) Electrical Conductivity Sensor Assembly for a Seed-Planting Implement

(71) Applicant: CNH Industrial America LLC, New Holland, PA (US)

(72) Inventor: Michael Christopher Conboy, Chicago, IL (US)

(21) Appl. No.: 18/924,772

(22) Filed: Oct. 23, 2024

(51) Int. Cl. G01N 33/24; A01B 49/06; A01C 5/06; A01C 7/08; A01C 7/20

(52) U.S. Cl. CPC G01N 33/245; A01B 49/06; A01C 5/068; A01C 7/08; A01C 7/203

(57) ABSTRACT

An electrical conductivity sensor assembly for a seed-planting implement includes a furrow firmer configured to shape a furrow formed in soil by the seed-planting implement, with the furrow firmer extending in a vertical direction from a top end to a bottom end. The furrow firmer defines a cavity at the bottom end. Furthermore, the assembly includes an electrode positioned within the cavity for use in determining the electrical conductivity of the soil. Additionally, the assembly includes a non-electrically conductive housing positioned between the electrode and the furrow firmer in the vertical direction.

### FIELD OF THE INVENTION

The present disclosure generally relates to seed-planting implements and, more particularly, to an electrical conductivity sensor assembly for a seed-planting implement.

### BACKGROUND OF THE INVENTION

Modern farming practices strive to increase yields of agricultural fields. In this respect, seed-planting implements are towed behind a tractor or other work vehicle to disperse seed throughout a field. For example, a seed-planting implement typically includes one or more furrow-forming tools, such as one or more disk openers, that excavate a furrow or trench in the soil. One or more dispensing devices of the seed-planting implement may, in turn, deposit the seeds into the furrow(s). After deposition of the seeds, a furrow-closing assembly may close the furrow in the soil, such as by pushing the excavated soil into the furrow.

The electrical conductivity of the soil within the field is an important parameter when controlling the operation of the seed-planting implement. In this respect, electrical conductivity sensors and sensor assemblies for seed-planting implements have been developed. While such sensors and sensor assemblies work well, further improvements are needed.

Accordingly, an improved electrical conductivity sensor assembly for a seed-planting implement would be welcomed in the technology.

### SUMMARY OF THE INVENTION

Aspects and advantages of the technology will be set forth in part in the following description or may be obvious from the description or may be learned through practice of the technology.

In one aspect, the present subject matter is directed to an electrical conductivity sensor assembly for a seed-planting implement. The assembly includes a furrow firmer configured to shape a furrow formed in soil by the seed-planting implement, with the furrow firmer extending in a vertical direction from a top end to a bottom end. The furrow firmer defines a cavity at the bottom end. Furthermore, the assembly includes an electrode positioned within the cavity for use in determining an electrical conductivity of the soil. Additionally, the assembly includes a non-electrically conductive housing positioned between the electrode and the furrow firmer in the vertical direction.

In another aspect, the present subject matter is directed to a row unit for a seed-planting implement. The row unit includes a row unit frame and a disk opener rotatably coupled to the row unit frame, with the disk opener configured to form a furrow within soil of a field as the seed-planting implement travels across the field. Moreover, the row unit includes a furrow firmer configured to shape the furrow, with the furrow firmer extending in a vertical direction from a top end to a bottom end. The furrow firmer defines a cavity at the bottom end. In addition, the row unit includes an electrode positioned within the cavity for use in determining an electrical conductivity of the soil. Furthermore, the assembly includes a non-electrically conductive housing positioned between the electrode and the furrow firmer in the vertical direction.

In a further aspect, the present subject matter is directed to a seed-planting implement including a toolbar and a first row unit supported on the toolbar. The first row unit includes a first furrow firmer configured to shape a first furrow formed by the first row unit, with the first furrow firmer extending in a vertical direction from a top end to a bottom end. The first furrow firmer defines a first cavity at the bottom end. Additionally, the first row unit includes a first electrode positioned within the first cavity and a first non-electrically conductive housing positioned between the first electrode and the first furrow firmer in the vertical direction. Moreover, the seed-planting implement includes a second row unit supported on the toolbar. The second row unit includes a second furrow firmer configured to shape a second furrow formed by the second row unit, with the second furrow firmer extending in the vertical direction from a top end to a bottom end. The second furrow firmer defines a second cavity at the bottom end. In addition, the second row unit includes a second electrode positioned within the second cavity and a second non-electrically conductive housing positioned between the second electrode and the second furrow firmer in the vertical direction. Furthermore, the seed-planting implement includes a third row unit supported on the toolbar. The third row unit includes a third furrow firmer configured to shape a third furrow formed by the third row unit, with the third furrow firmer extending in the vertical direction from a top end to a bottom end. The third furrow firmer defines a third cavity at the bottom end. Additionally, the third row unit includes a third electrode positioned within the third cavity and a third non-electrically conductive housing positioned between the third electrode and the third furrow firmer in the vertical direction. Moreover, the seed-planting implement includes a fourth row unit supported on the toolbar. The fourth row unit includes a fourth furrow firmer configured to shape a fourth furrow formed by the fourth row unit, with the fourth furrow firmer extending in the vertical direction from a top end to a bottom end. The fourth furrow firmer defines a fourth cavity at the bottom end. In addition, the fourth row unit includes a fourth electrode positioned within the fourth cavity and a fourth non-electrically conductive housing positioned between the fourth electrode and the fourth furrow firmer in the vertical direction.

These and other features, aspects, and advantages of the present technology will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present technology, including the best mode thereof, directed to one of ordinary skill in the art, is set forth in the specification, which refers to the appended figures, in which:

FIG. 1 illustrates a perspective view of one embodiment of a seed-planting implement in accordance with aspects of the present subject matter;

FIG. 2 illustrates a side view of one embodiment of a row unit of a seed-planting implement in accordance with aspects of the present subject matter;

FIG. 3 illustrates a cross-sectional view of one embodiment of an electrical conductivity sensor assembly in accordance with aspects of the present subject matter; and

FIG. 4 illustrates a diagrammatic view of one embodiment of a seed-planting implement in accordance with aspects of the present subject matter, particularly illustrating implementation of an electrical conductivity sensor assembly on several row units of the seed-planting implement.

Repeat use of reference characters in the present specification and drawings is intended to represent the same or analogous features or elements of the present technology.

### DETAILED DESCRIPTION OF THE DRAWINGS

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield still a further embodiment. Thus, it is intended that the present invention covers such modifications and variations as come within the scope of the appended claims and their equivalents.

As used herein, the term “and/or,” when used in a list of two or more items, means that any one of the listed items can be employed by itself, or any combination of two or more of the listed items can be employed. For example, if a composition or assembly is described as containing components A, B, and/or C, the composition or assembly can contain A alone; B alone; C alone; A and B in combination; A and C in combination; B and C in combination; or A, B, and C in combination.

In general, the present subject matter is directed to an electrical conductivity sensor assembly for a seed-planting implement. As will be described below, the seed-planting implement includes a furrow firmer configured to shape the furrow formed in the soil by the row unit. In this respect, the furrow firmer extends in a vertical direction from a top end to a bottom end such that the furrow firmer defines a cavity at the bottom end.

Additionally, the electrical conductivity sensor assembly includes an electrode and a non-electrically conductive housing. Specifically, in several embodiments, the electrode is positioned within the cavity of the furrow firmer for use in determining the electrical conductivity of the soil. Moreover, the non-electrically conductive housing is positioned between the electrode and the furrow firmer in the vertical direction. In this respect, the non-electrically conductive housing electrically isolates the electrode from the furrow firmer. For example, the electrode may be a metallic material, such as a metallic strip, and the non-electrically conductive housing may be formed of a polymeric material.

The disclosed electrical conductivity sensor improves the operation of the seed-planting implement. More specifically, as described above, the disclosed electrical conductivity sensor includes a non-electrically conductive housing electrically, which isolates the electrode from the furrow firmer. In this respect, the disclosed electrical conductivity sensor assembly is positioned within a furrow firmer of the seed-planting implement. Thus, the disclosed electrical conductivity sensor assembly does not negative affect furrow closing operation unlike conventional electrical conductivity sensors that bolt onto the seed-planting implement behind the furrow firmer. This, in turn, improves the agricultural performance of the field.

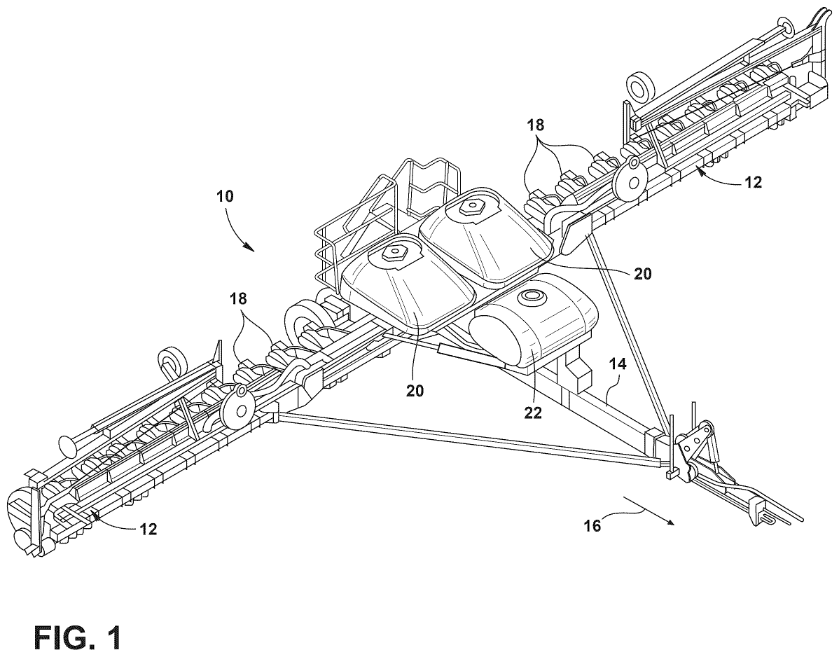

Referring now to drawings, FIG. 1 illustrates a perspective view of one embodiment of a seed-planting implement 10. In the illustrated embodiment, the seed-planting implement 10 is configured as a planter. However, in alternative embodiments, the seed-planting implement 10 may be configured as a seeder, a strip-tiller, a side-dresser, or any other suitable agricultural implement that deposits seeds into a field.

As shown in FIG. 1, the seed-planting implement 10 may include a laterally extending toolbar 12. More specifically, the toolbar 12 is connected at its middle to a forwardly extending tow bar 14 to allow the seed-planting implement 10 to be towed by a work vehicle (not shown), such as an agricultural tractor, in a direction of travel 16. In this respect, the toolbar 12 is generally configured to support a plurality of seed planting units or row units 18. Each row unit 18, in turn, is configured to deposit seeds at a desired depth beneath the soil surface and with a desired seed spacing as the seed-planting implement 10 travels across the field in the direction of travel 16, thereby establishing rows of planted seeds. In some embodiments, the bulk of the seeds to be planted may be stored in one or more hoppers or seed tanks 20. Thus, as seeds are planted by the row units 18, a pneumatic distribution system may distribute additional seeds from the seed tanks 20 to the individual row units 18. Additionally, one or more fluid tanks 22 may store agricultural fluids, such as insecticides, herbicides, fungicides, fertilizers, and/or the like. These fluids, in turn, may be supplied to the row units 18 for spraying onto the seeds during planting.

For purposes of illustration, only a portion of the row units 18 of the seed-planting implement 10 has been shown in FIG. 1. In general, the seed-planting implement 10 may include any number of row units 18, such as 6, 8, 12, 16, 24, 32, or 36 row units. In addition, the lateral spacing between row units 18 may be selected based on the type of crop being planted. For example, the row units 18 may be spaced approximately 30 inches from one another for planting corn, and approximately 15 inches from one another for planting soybeans.

The configuration of the seed-planting implement 10 described above and shown in FIG. 1 is provided only to place the present subject matter in an exemplary field of use. Thus, the present subject matter may be readily adaptable to any manner of seed-planting implement configuration.

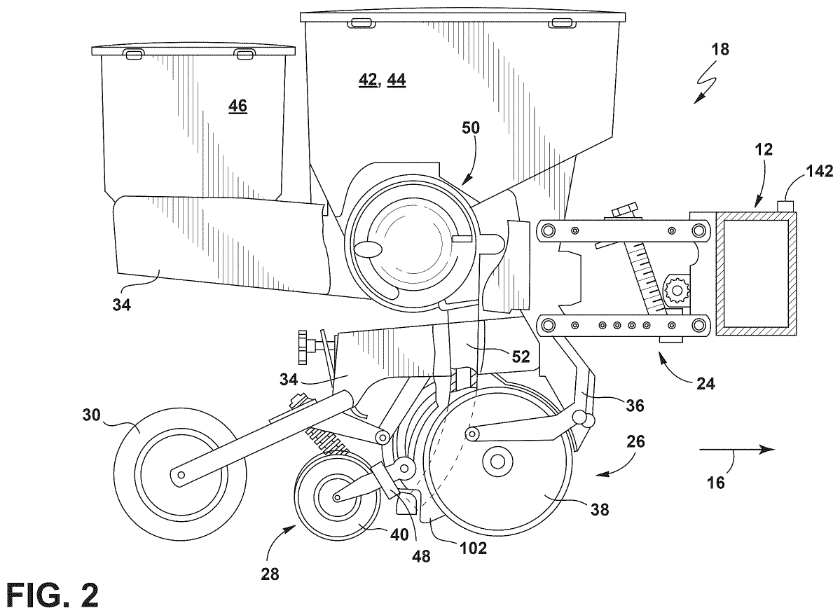

FIG. 2 illustrates a side view of one embodiment of a row unit 18. As shown, the row unit 18 includes a linkage assembly 24 configured to mount the row unit 18 to the toolbar 12 of the seed-planting implement 10. Furthermore, the row unit 18 also includes a row unit frame 34. In this respect, the row unit 18 may include a furrow opening assembly 26, a furrow closing assembly 28, and a press wheel 30 supported on or otherwise coupled row unit frame 34. In general, the furrow opening assembly 26 may include a gauge wheel (not shown) operatively coupled to the row unit frame 34 via a support arm 36. Additionally, the opening assembly 26 may also include one or more disk openers 38 rotatably coupled to the row unit frame 34. Moreover, the row unit 18 includes a furrow firmer 102 coupled to the row unit frame 34. The gauge wheel is not shown in FIG. 2 to better illustrate the disk opener(s) 38 and furrow firmer 102. The disk opener(s) 38 is configured to form or otherwise excavate a furrow or trench within the soil of a field as the seed-planting implement 10 (FIG. 1) travels across the field in the direction of travel 16. In this respect, the furrow firmer 102 is configured to shape the furrow formed in soil by the disk opener(s) 38 and firm the walls of such firm to prevent premature collapse of the furrow. In addition, the gauge wheel is configured to roll along or otherwise engage the surface of the field such that the position of the gauge wheel relative to the row unit frame 34 sets the depth of the furrow being excavated. Furthermore, as shown, the furrow closing assembly 28 may include a closing disk(s) 40 configured to close or collapse the furrow after seeds have been deposited therein. Thereafter, the press wheel 30 may roll over the closed furrow to firm the soil over the seed and promote favorable seed-to-soil contact.

Additionally, as shown in FIG. 2, the row unit 18 may include one or more seed hoppers 42, 44 and a fluid tank 46 supported on the row unit frame 34. In general, the seed hopper(s) 42, 44 may be configured to store seeds received from the seed tanks 20, which are to be deposited within the furrow as the row unit 18 travels across the field. For instance, in several embodiments, the row unit 18 may include a first seed hopper 42 configured to store seeds of a first seed type and a second hopper 44 configured to store seeds of a second seed type. However, both seed hoppers 42, 44 may be configured to store the same type of seeds. Furthermore, the fluid tank 46 may be configured to store fluid received from the fluid tank 22 (FIG. 1), which is to be sprayed onto the seeds dispensed from the seed hoppers 42, 44. For example, a sprayer assembly 48 mounted on the row unit frame 34 may be configured to spray the fluid stored in the fluid tank 22 onto the seeds.

Moreover, the row unit 18 may include a seed meter 50 supported on the row unit frame 34. In general, the seed meter 50 is configured to uniformly release seeds received from the seed hopper(s) 42, 44 for deposition within the furrow. For instance, in one embodiment, the seed meter 50 may be coupled to a suitable vacuum source (e.g., a blower powered by a motor and associated tubing or hoses) configured to generate a vacuum or negative pressure that attaches the seeds to a rotating seed disk of the seed meter 50, which controls the rate at which the seeds are output from the seed meter 50 to an associated seed tube 52. As shown in FIG. 2, the seed tube 52 may extend vertically from the seed meter 50 toward the ground to facilitate delivery of the seeds discharged from the seed meter 50 to the furrow.

The configuration of the row unit 18 described above and shown in FIG. 2 is provided only to place the present subject matter in an exemplary field of use. Thus, the present subject matter may be readily adaptable to any manner of seed planting unit configuration.

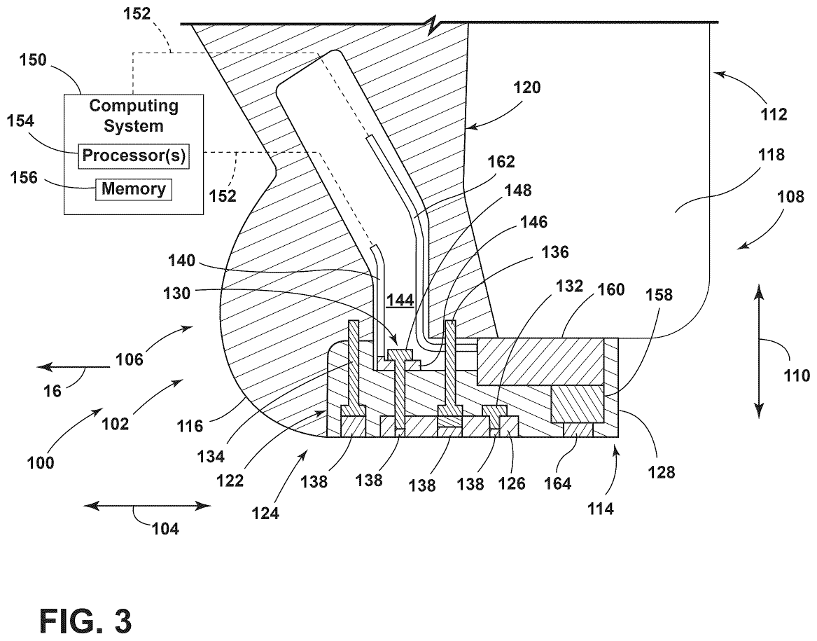

FIG. 3 illustrates a cross-sectional view of one embodiment of an electrical conductivity sensor assembly 100 for a seed-planting implement. In general, the electrical conductivity sensor assembly 100 will be described herein with reference to the seed-planting implement 10 and the row unit 18 described above with reference to FIGS. 1 and 2. However, the disclosed electrical conductivity sensor assembly 100 can generally be utilized with seed-planting implements having any other suitable implement configuration and/or row units having any other suitable row unit configuration.

As shown in FIG. 3, the electrical conductivity sensor assembly 100 includes the furrow firmer 102 of the row unit 18. More specifically, the furrow firmer 102 extends in a longitudinal direction 104 from a forward end 106 to an aft end 108, with the longitudinal direction 104 extending generally parallel to the direction of travel 16. Furthermore, the furrow firmer 102 extends in a vertical direction 110 from a top end 112 to a bottom end 114, with the vertical direction 110 extending generally perpendicular to the longitudinal direction 104. In some embodiments, the furrow firmer 102 includes a body 116 (e.g., a metallic casting) and a sleeve 118 (e.g., formed of sheet metal) coupled to an aft end 120 of the body 116. Additionally, the furrow firmer 102 defines a cavity 122 at the bottom end 114. For example, in the illustrated embodiment, the cavity 122 is defined at a bottom end 124 of the body 116. As will be described below, additional components of the electrical conductivity sensor assembly 100 are positioned within the cavity 122, thereby allowing for the determination of the electrical conductivity of the soil while not negatively impacting the furrow closing operation being performed by the closing disk(s) 40 (FIG. 2).

Furthermore, the electrical conductivity sensor assembly 100 includes an electrode 126 positioned within the cavity 122. In general, the electrode 126 extends in the longitudinal direction 104 between the forward and aft ends 106, 108 of the furrow firmer 102. Moreover, the electrode 126 is positioned such that the electrode 126 contacts the soil forming the bottom surface of the furrow as the row unit 18 travels across the field in the direction of travel 16. In this respect, and as will be described below, the electrode 126 is used in determining the electrical conductivity of the soil forming the furrow. Thus, in several embodiments, the electrode 126 is formed of a metallic material. For example, in some embodiments, the electrode 126 is configured as a metallic strip or plate.

Additionally, the electrical conductivity sensor assembly 100 includes a non-electrically conductive housing 128. As shown, the non-electrically conductive housing 128 is positioned within the cavity 122. Moreover, the non-electrically conductive housing 128 is positioned between the electrode 126 and at least a portion of the furrow firmer 102 in the vertical direction 110. In this respect, the non-electrically conductive housing 128 electrically isolates the electrode 126 from the furrow firmer 102. Thus, the non-electrically conductive housing 128 allows the electrode 126 to be positioned within the furrow firmer 102 without shorting on the body 116 or the sleeve 118.

The non-electrically conductive housing 128 may be formed out of any suitable non-electrically conductive or otherwise electrically insulative material. For example, in some embodiments, the non-electrically conductive housing 128 may be formed of a polymeric material.

Moreover, the electrode 126 and the non-electrically conductive housing 128 may be mechanically coupled to the furrow firmer 102 in any suitable manner. More specifically, in some embodiments, the electrode 126 may be mechanically coupled to the non-electrically conductive housing 128 via one or more fasteners. For example, in the illustrated embodiment, the electrode 126 is mechanically coupled to the non-electrically conductive housing 128 via a first fastener 130 and a second fastener 132. Similarly, the non-electrically conductive housing 128 may be mechanically coupled to the furrow firmer 102 via one or more fasteners. For example, in the illustrated embodiment, the non-electrically conductive housing 128 is mechanically coupled to the furrow firmer 102 (e.g., the body 116 of the furrow firmer 102) via a third fastener 134 and a fourth fastener 136. In some embodiments, caps 138 may be placed in the holes in which the first, second, third, and/or fourth fasteners 130, 132, 134, 136 are received to prevent soil accumulation therein.

In addition, the electrical conductivity sensor assembly 100 includes a wire 140 and a circuit board 142 (FIG. 2) positioned on the toolbar 12. More specifically, as shown in FIG. 3, the furrow firmer 102 and/or the non-electrically conductive housing 128 may define a passage 144. In this respect, the wire 140 may be routed at least partially through the passage 144. For example, one end of the wire 140 may be electrically coupled to the circuit board 142, while a terminal 146 positioned within the cavity 122 may be mechanically coupled to the opposing end of the wire 140. In this respect, one of the fasteners coupling the electrode 126 and the non-electrically conductive housing 128 may electrically couple the terminal 146 and the electrode 126. For example, in the illustrated embodiment, at least a portion of the terminal 146 is positioned between a head 148 of the fastener 130 and the non-electrically conductive housing 128 in the vertical direction 110. Thus, electric current may flow from the electrode 126 through the fastener 130 and into the terminal 146 before flowing through the wire 140 to the circuit board 142. Alternatively, electric current may flow in the opposite direction.

Moreover, the electrical conductivity sensor assembly 100 includes a computing system 150 communicatively coupled to one or more components of the electrical conductivity sensor assembly 100, the row unit 18, and/or the seed-planting implement 10. For instance, in some embodiments, the computing system 150 may be communicatively coupled to the circuit board 142 via a communicative link 152. Alternatively, the circuit board 142 may be part of the computing system 150. As such, the computing system 150 may be configured to receive electric current or other data from the electrode 126 (or the circuit board 142) via the wire 140 and/or the wrie 140, which may form part of the communicative link 152. Such electric current or data may generally be indicative of the electrical conductivity of the soil within the field. In addition, the computing system 150 may be communicatively coupled to any other suitable components of the electrical conductivity sensor assembly 100, the row unit 18, and/or the seed-planting implement 10, such as any other sensor(s) positioned within the cavity 122.

In general, the computing system 150 may include one or more processor-based devices, such as a given controller or computing device or any suitable combination of controllers or computing devices. Thus, in several embodiments, the computing system 150 may include one or more processor(s) 154 and associated memory device(s) 156 configured to perform a variety of computer-implemented functions. As used herein, the term “processor” refers not only to integrated circuits referred to in the art as being included in a computer, but also refers to a controller, a microcontroller, a microcomputer, a programmable logic circuit (PLC), an application specific integrated circuit, and other programmable circuits. Additionally, the memory device(s) 156 of the computing system 150 may generally comprise memory element(s) including, but not limited to, a computer-readable medium (e.g., random access memory RAM)), a computer-readable non-volatile medium (e.g., a flash memory), a floppy disk, a compact disk-read only memory (CD-ROM), a magneto-optical disk (MOD), a digital versatile disk (DVD) and/or other suitable memory elements. Such memory device(s) 156 may generally be configured to store suitable computer-readable instructions that, when implemented by the processor(s) 154, configure the computing system 150 to perform various computer-implemented functions. In addition, the computing system 150 may also include various other suitable components, such as a communications circuit or module, one or more input/output channels, a data/control bus, and/or the like.

The various functions of the computing system 150 may be performed by a single processor-based device or may be distributed across any number of processor-based devices, in which instance such devices may be considered to form part of the computing system 150. For instance, the functions of the computing system 150 may be distributed across multiple application-specific controllers or computing devices (e.g., the circuit board 142 may be part of the computing system 150).

Furthermore, as indicated above, other sensors may be positioned within the cavity 122 defined by the furrow firmer. For example, in some embodiments, an optical sensor 158 may be positioned within the cavity 122. In general, the optical sensor 158 may be configured to generate data indicative of one or more other parameters of the soil, such as the organic matter content of the soil. More specifically, the optical sensor 158 may be coupled to the non-electrically conductive housing 128. For example, in one embodiment, the optical sensor 158 may be coupled to a circuit board 160 that, in turn, is coupled (e.g., potted) to the non-electrically conductive housing 128. A wire 162 may be coupled (e.g., soldered) to the circuit board 160 and routed through the passage 144 for eventual direct or indirect coupling to the computing system 150. In alternative embodiments, the circuit board 160 may be considered part of the computing system 150 and the wire 162 may be part of the communicative link 152. Additionally, a lens 164 may be positioned within the cavity 122 and coupled (e.g., adhesively coupled) to the non-electrically conductive housing 128. As such, the optical sensor 158 has a field of view through the lens 164 that is directed at the bottom surface of the furrow. That is, the optical sensor 158 can view the soil defining the bottom surface of the furrow through the lens 164. In this respect, the optical sensor 158 is at least partially positioned between the circuit board 160 and the lens 164 in the vertical direction 110. However, in alternative embodiments, the optical sensor 158 may be omitted, and/or other types of sensors may be positioned within the cavity 122.

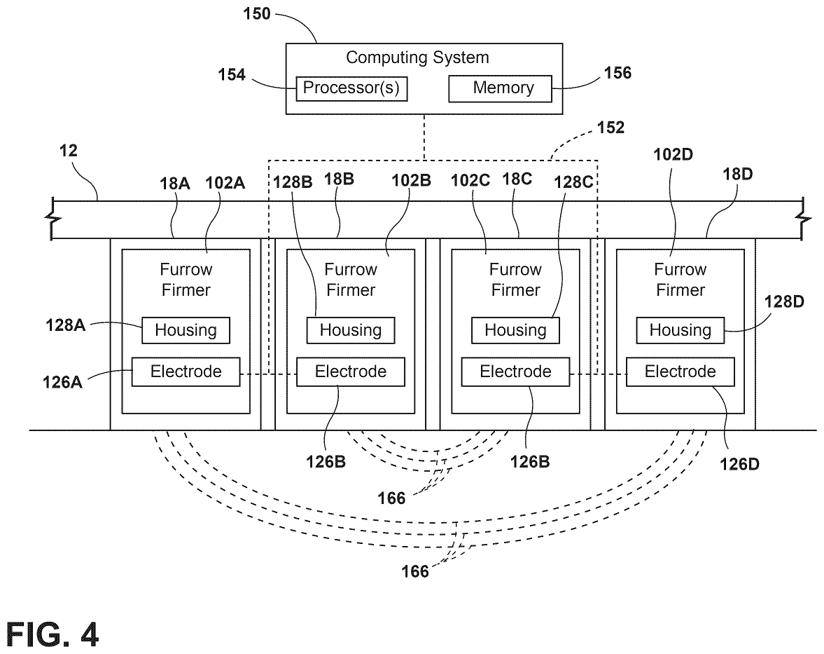

FIG. 4 illustrates a diagrammatic view of one embodiment of a seed-planting implement 10. Specifically, FIG. 4 the illustrates implementation of four electrical conductivity sensor assemblies on four different row units of the seed-planting implement 10. As will be described below, the use of four electrical conductivity sensor assemblies can allow for determination of the electrical conductivity of the soil within the field.

As shown, the seed-planting implement 10 includes first, second, third, and fourth row units 18A-D. In this respect, the first row unit 18A includes a first furrow firmer 102A defining a first cavity in which a first electrode 126A and a first non-electrically conductive housing 128A are positioned. Similarly, the second row unit 18B includes a second furrow firmer 102B defining a second cavity in which a second electrode 126B and a second non-electrically conductive housing 128B are positioned. Moreover, the third row unit 18C includes a third furrow firmer 102C defining a third cavity in which a third electrode 126C and a third non-electrically conductive housing 128C are positioned. In addition, the fourth row unit 18D includes a fourth furrow firmer 102D defining a fourth cavity in which a fourth electrode 126D and a fourth non-electrically conductive housing 128D are positioned. Thus, the seed-planting implement 10 includes four electrical conductivity sensor assemblies 100. The seed-planting implement 10 may include additional row units not having electrical conductivity sensor assemblies incorporated therein.

As indicated above, the computing system 150 is configured to use the first, second, third, and fourth electrodes 126A-D to determine the electrical conductivity of soil within the field across which the seed-planting implement 10 is traveling. More specifically, the computing system 150 is electrically coupled to the first electrode 126A, the second electrode 126B, the third electrode 126C, and the fourth electrode 126D. Furthermore, the first electrode 126A, the second electrode 126B, the third electrode 126C, and the fourth electrode 126D are configured to transmit an electric current (indicated by dashed lines 166) through the soil. For example, the computing system 150 may control the operation the of a power source or associated switches such that electric power flow through one of the first or fourth electrode 126A, 126D through the soil to the other of the first or fourth electrodes 126A, 126D. In this respect, the computing system 150 may be configured to determine the electrical conductivity of the soil based on a voltage detected between the second electrode 126B and the third electrode 126C. For example, the computing system 150 may include or otherwise be coupled to a voltmeter or voltage sensing device that measures the voltage or the resistance between the second and third electrodes 126B, 126C. This voltage or resistance is, in turn, indicative of the electrical conductivity of the soil. For example, the computing system 150 include a look-up stored within its memory device(s) 156 correlating the voltage between the second and third electrodes 126B, 126C with an electrical conductivity value for the soil.

This written description uses examples to disclose the technology, including the best mode, and also to enable any person skilled in the art to practice the technology, including making and using any devices or systems and performing any incorporated methods. The patentable scope of the technology is defined by the claims and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. An electrical conductivity sensor assembly for a seed-planting implement, the electrical conductivity sensor assembly comprising:
a furrow firmer configured to shape a furrow formed in soil by the seed-planting implement, the furrow firmer extending in a vertical direction from a top end to a bottom end, the furrow firmer defining a cavity at the bottom end;
an electrode positioned within the cavity for use in determining an electrical conductivity of the soil; and
a non-electrically conductive housing positioned between the electrode and the furrow firmer in the vertical direction.

2. The electrical conductivity sensor assembly of claim 1, wherein the non-electrically conductive housing electrically isolates the electrode from the furrow firmer.

3. The electrical conductivity sensor assembly of claim 1, wherein at least one of the furrow firmer or the non-electrically conductive housing further defines a passage, the assembly further comprising:
a wire extending through the passage and electrically coupled to the electrode.

4. The electrical conductivity sensor assembly of claim 3, further comprising:
a terminal positioned within the cavity and mechanically coupled to an end of the wire.

5. The electrical conductivity sensor assembly of claim 4, further comprising:
a fastener mechanically coupling the electrode and the non-electrically conductive housing, the fastener further electrically coupling the terminal and the electrode.

6. The electrical conductivity sensor assembly of claim 5, wherein at least a portion of the terminal is positioned between a head of the fastener and the non-electrically conductive housing in the vertical direction.

7. The electrical conductivity sensor assembly of claim 1, wherein the non-electrically conductive housing is mechanically coupled to the furrow firmer via one or more fasteners.

8. The electrical conductivity sensor assembly of claim 1, wherein the electrode is mechanically coupled to the non-electrically conductive housing via one or more fasteners.

9. The electrical conductivity sensor assembly of claim 1, wherein the non-electrically conductive housing is formed of a polymeric material and the electrode is formed of a metallic material.

10. The electrical conductivity sensor assembly of claim 1, wherein the electrode is a metallic strip.

11. A row unit for a seed-planting implement, the row unit comprising:
a row unit frame;
a disk opener rotatably coupled to the row unit frame, the disk opener configured to form a furrow within soil of a field as the seed-planting implement travels across the field;
a furrow firmer coupled to the row unit frame, the furrow firmer configured to shape the furrow, the furrow firmer extending in a vertical direction from a top end to a bottom end, the furrow firmer defining a cavity at the bottom end;
an electrode positioned within the cavity for use in determining an electrical conductivity of the soil; and
a non-electrically conductive housing positioned between the electrode and the furrow firmer in the vertical direction.

12. The row unit of claim 11, wherein the non-electrically conductive housing electrically isolates the electrode from the furrow firmer.

13. The row unit of claim 11, wherein at least one of the furrow firmer or the non-electrically conductive housing further defines a passage, the assembly further comprising:
a wire extending through the passage and electrically coupled to the electrode.

14. The row unit of claim 13, further comprising:
a terminal positioned within the cavity and mechanically coupled to an end of the wire.

15. The row unit of claim 14, further comprising:
a fastener mechanically coupling the electrode and the non-electrically conductive housing, the fastener further electrically coupling the terminal and the electrode.

16. The row unit of claim 15, wherein at least a portion of the terminal is positioned between a head of the fastener and the non-electrically conductive housing in the vertical direction.

17. The assembly of claim 1, further comprising:
an optical sensor positioned within the cavity.

18. A seed-planting implement, comprising:
a toolbar;
a first row unit supported on the toolbar, the first row unit comprising:
a first furrow firmer configured to shape a first furrow formed by the first row unit, the first furrow firmer extending in a vertical direction from a top end to a bottom end, the first furrow firmer defining a first cavity at the bottom end;
a first electrode positioned within the first cavity; and
a first non-electrically conductive housing positioned between the first electrode and the first furrow firmer in the vertical direction;
a second row unit supported on the toolbar, the second row unit comprising:
a second furrow firmer configured to shape a second furrow formed by the second row unit, the second furrow firmer extending in the vertical direction from a top end to a bottom end, the second furrow firmer defining a second cavity at the bottom end;
a second electrode positioned within the second cavity; and
a second non-electrically conductive housing positioned between the second electrode and the second furrow firmer in the vertical direction;
a third row unit supported on the toolbar, the third row unit comprising:
a third furrow firmer configured to shape a third furrow formed by the third row unit, the third furrow firmer extending in the vertical direction from a top end to a bottom end, the third furrow firmer defining a third cavity at the bottom end;
a third electrode positioned within the third cavity; and
a third non-electrically conductive housing positioned between the third electrode and the third furrow firmer in the vertical direction; and
a fourth row unit supported on the toolbar, the fourth row unit comprising:
a fourth furrow firmer configured to shape a fourth furrow formed by the fourth row unit, the fourth furrow firmer extending in the vertical direction from a top end to a bottom end, the fourth furrow firmer defining a fourth cavity at the bottom end;
a fourth electrode positioned within the fourth cavity; and
a fourth non-electrically conductive housing positioned between the fourth electrode and the fourth furrow firmer in the vertical direction.

19. The seed-planting implement of claim 18, wherein the first electrode, the second electrode, the third electrode, and the fourth electrode are configured to transmit an electric current through the soil.

20. The seed-planting implement of claim 19, further comprising:
a computing system electrically coupled to the first electrode, the second electrode, the third electrode, and the fourth electrode,
wherein the computing system is configured to determine an electrical conductivity of the soil based on a voltage or a resistance detected between the second electrode and the third electrode.
